(19) United States
(12) Patent Application Publication
(10) Pub. No.: US 2007/0243549 A1
(43) Pub. Date: Oct. 18, 2007

(54) ENRICHMENT OF CIRCULATING FETAL DNA

(75) Inventor: Farideh Z. Bischoff, Sugar Land, TX (US)

Correspondence Address: COOLEY GODWARD KRONISH LLP;ATTN: Patent Group, Suite 500, 1200 - 19th Street, NW, WASHINGTON, DC, 20036-2402, US

(73) Assignee: BIOCEPT, INC., San Diego, CA (US)

(21) Appl. No.: 11/734,224

(22) Filed: Apr. 11, 2007

Related U.S. Application Data
(60) Provisional application No. 60/744,740, filed on Apr. 12, 2006.

(51) Int. Cl. C12Q 1/68; C12P 19/34; C07K 16/18

(52) U.S. Cl. 435/6; 435/91.2; 530/388.1

(57) ABSTRACT

A non-invasive screening or diagnostic method for determining the likelihood of a fetus with a genetic abnormality or a potential pregnancy complication, which utilizes a liquid blood sample from a pregnant woman. Antibodies specific to a section of histone 3.1 which is exposed to a far greater extent in chromatin of fetal origin than in chromatin of maternal origin are used to sequester and isolate such fetal nucleosomes including the associated fetal DNA. Following isolation/enrichment of such fetal DNA, genetic analysis is carried out using known molecular diagnostics.

### RELATED U.S. PATENT APPLICATIONS

This application claims the benefit of U.S. Provisional Application No. 60/744,740 filed Apr. 12, 2006, the disclosure of which is incorporated herein by reference in its entirety.

### FIELD OF THE INVENTION

The present invention relates to a method for distinguishing fetal DNA from maternal DNA which are concurrently present in maternal plasma/serum. More particularly, the present invention relates to a method of definitive, non-invasive, prenatal, genetic testing of fetal DNA in a pregnant woman early in pregnancy. The ability to distinguish fetal from maternal DNA will permit enrichment of fetal DNA and thus development of more definitive, non-invasive, molecular DNA screening and diagnostic tests for potential prenatal genetic disorders. Obtaining such fetal DNA from a maternal blood sample will potentially enable diagnosis of heritable single gene mutations as well as chromosomal aneuploidy (e.g., Trisomy 21) in the fetus. Such enriched fetal DNA can be subjected to molecular DNA sequencing and/or single nucleotide polymorphic marker analysis for detection of qualitative (gene mutation) and/or quantitative (aneuploidy detection) fetal genetic alteration.

### BACKGROUND OF THE INVENTION

Chromosomal abnormalities occur in 0.1% to 0.2% of live births. Among these, the most common, clinically significant abnormality is Down syndrome (Trisomy 21). In addition, single gene mutations account for a small proportion of genetic alterations as well (˜1 in 12,000 live births). There are currently both screening and diagnostic tests for chromosomal abnormalities, but unfortunately, all of them have serious limitations. The screening tests suffer from less than desirable sensitivity and/or specificity, and the diagnostic tests involve small but significant risks to the fetus and mother in obtaining the needed fetal cells.

Cultured fetal cells obtained through an invasive procedure can be subjected to molecular DNA and/or cytogenetic analysis, permitting diagnosis of fetal single gene mutations and/or chromosomal abnormalities, including aneuploidies, such as Trisomy 13, Trisomy 18, Klinefelter syndrome, XYY, Turner syndrome and Down syndrome (Trisomy 21). A major disadvantage to this approach is that an invasive procedure, either amniocentesis or chorionic villus sampling (CVS), is required to obtain fetal cells, and such presents three problems: (1) risk to both the fetus and the mother, (2) delay in diagnosis, and (3) cost. Because amniocentesis and CVS are both invasive procedures, there is a small but significant risk to the fetus and a slight risk of infection for the mother. Moreover, both of these tests have specific windows of time in which they may be carried out. Amniocentesis is generally done at 15 weeks or greater gestation. Chorionic villus sampling is done at 9-12 weeks gestation. Earlier diagnosis afforded by CVS is advantageous because of reduced emotional stress on the parents and medical advantages associated with an early termination of pregnancy should the parents so choose. However, earlier diagnosis entails an increased risk to the fetus.

The risk of fetal loss, although small, is significant. It is generally quoted that there is about a 0.5% risk of fetal loss as a consequence of a second-trimester (16 week) amniocentesis. The risk associated with CVS is somewhat greater. For women under age 35 without a predisposing factor, the risk of fetal loss due to amniocentesis is felt to be greater than the incidence of Down syndrome; thus, a diagnostic test is often only recommended for women 35 or over unless there is another predisposing factor. The most common predisposing factor is a positive screening test. Although the incidence of Down syndrome increases rapidly with increasing age for women over 35, it must be remembered that women under 35 account for about 80% of the overall number of Down syndrome births. At age 35, the incidence may be about one in 200 live births; it increases to about one in 46 at age 45. Although the risk of Down syndrome (as well as other chromosome abnormalities) is greatly increased, the consequences of a fetal loss due to amniocentesis are also much greater, since these older women may not be able to achieve another pregnancy.

Because of the risks associated with the prenatal diagnostic tests currently available, a large amount of effort has been dedicated towards developing more effective screening tests. Whereas the diagnostic test is a highly accurate and sensitive way of detecting chromosomal aneuploidies, the screening tests that are currently available provide only some indication of the likelihood of whether or not a fetus is affected with Down syndrome or another chromosomal aneuploidy. A negative result from a screening test does not necessarily mean that the child will be unaffected (only that there is a lower risk); moreover, a positive result must be followed up by an invasive diagnostic test to be meaningful. Because of the relatively low specificity of the current screening tests and the requirement that positive tests be validated by a diagnostic cytogenetic test, a large number of normal pregnancies continue to be jeopardized by amniocentesis.

There are two types of screening tests generally now available: a blood test conducted on the mother, and an ultrasound test conducted on the fetus. The blood test is generally done in the second trimester, typically between 15 and 20 weeks gestation. In this test, a blood sample is taken from the mother and the levels of one, two, three or four biochemical markers are determined. This test is referred to as a “triple screen” if three markers are determined, or a “quad screen” if four markers are determined. The results of these tests also serve as a screening test for Trisomy 18 and for neural tube defects.

The use of a triple screen for pregnant women under age 35 may be the current standard of practice covered by many insurance companies. The markers that are measured in the triple screen are alpha-fetoprotein, chorionic gonadotropin, and unconjugated estriol. Recently, a fourth biochemical marker, inhibin-A, has been added to the triple screen to form the “quad screen.”

The triple screen has been in use for a number of years, and a considerable amount of data on the sensitivity and specificity of the test has been accumulated. Sensitivity and specificity vary with the age of the mother and with the cutoff criteria used by the various investigators. Generally, out of 1000 women tested, about 100 will test positive, i.e., meaning a recommendation will result to follow up with amniocentesis for a cytogenetic study. Of this 100, only two or three will actually have a fetus with Down syndrome. Of the 900 who test negative, about two will have a child with Down syndrome. Thus, many providers do not believe that this test truly provides a woman with any greatly increased assurance she is carrying a child without Down syndrome; instead, it is felt that it subjects many couples to the emotional stress associated with receiving a positive test and also subjects many normal fetuses to the risks of amniocentesis.

Second-trimester ultrasound screening has alternatively become a routine part of prenatal care in many practices, and several sonographic markers have been associated with chromosomal abnormalities. However, review of studies conducted for over a decade found that, in the absence of associated fetal abnormalities, the sensitivity of these markers was low and that there was a relatively high false positive rate in detecting Down syndrome.

U.S. Pat. No. 5,252,489 discloses a screening test for Down syndrome and perhaps other chromosomal anomalies to determine whether a pregnant woman's risk of carrying a fetus with Down syndrome warrants further testing. The test procedure can utilize a few drops of blood from a prick at the tip of a finger, an earlobe or the like, which drops are collected on a piece of filter paper or the like. The test relies upon comparison of the level of free beta HCG in the dried blood spot against reference values of the level of free beta HCG accumulated by testing women during similar gestational periods who then experienced either normal childbirth or a child or fetus diagnosed with a chromosomal anomaly such as Down syndrome. Based upon this comparison, a risk assessment is made to allow the pregnant woman to decide whether she should then undergo diagnostic testing or whether the risk appears to be so low that further testing is felt to be unwarranted. Although the concept of such screening is good, it may not be more effective than the previously described triple screen and/or quad screen, and it has not achieved wide acceptance because the results have not been shown to be sufficiently accurate to provide parents with any greatly increased assurance of whether the fetus is or is not affected with Down syndrome.

It has been realized for some time now that there are fetal cells which are present in the mother's blood, and that these cells present a potential source of fetal chromosomes for prenatal DNA-based diagnostics. Because these cells appear very early in the pregnancy, they could form the basis of an accurate, noninvasive, first trimester test. A number of methods for isolating these cells have been proposed, and several laboratories are exploring methods to isolate fetal cells from the mother's blood, and to use the DNA from these cells for prenatal diagnosis.

One approach that has been used to achieve enrichment of fetal cells within a maternal blood sample utilizes antibodies (Abs) specific for a particular fetal cell type to couple to and capture fetal cells or to label fetal cells. In U.S. Pat. No. 5,641,628, fetal-specific, detectably labeled antibodies are used to label fetal cells and, when bound to these fetal cells, facilitate separation of these cells from maternal components by flow cytometry. Another method of separating target cells from heterogenous cell populations uses beds of particles, e.g., beads, which carry sequestering agents in the form of antibodies (Abs) that are directed at a ligand carried on the exterior surface of the target cells. The bodily fluid may be caused to flow through a stationary bed of such beads, or a group or bed of such beads may be caused to move, as by gravity, through a sample of the bodily fluid in question. U.S. Pat. No. 5,766,843 teaches the bonding of anti-CD45 antibodies to the exterior surface of solid supports, such as magnetic beads, which are then used to selectively bind to white blood cells. U.S. Published Patent Application No. 2004/0018509 mentions the use of commercially available “Dynabeads” having magnetic cores, which are coated with antibodies, for removing placenta-derived trophoblast cells in the blood of pregnant women.

Such an effective test would have compelling advantages over those that are currently available as it would be noninvasive and could be done very early in pregnancy. The major problem that must be overcome to render such an approach feasible is the achievement of effective isolation of the fetal cells, exclusive of the maternal cells, which fetal cells are present in the mother's blood in only very small numbers. Unfortunately, clinical feasibility has not yet been demonstrated for any of these methods.

An alternative and perhaps more attractive approach to obtaining fetal cells would be to use circulating, cell-free, fetal DNA in maternal blood. Circulating nucleic acids in plasma were first observed over 50 years ago; however, it was not until 1970, when new molecular techniques emerged, that DNA fragments in the plasma were found to be associated with tumors among patients with various types of cancer. In the last decade, there have been over 1500 publications and clinical trials initiated to address the utility and clinical applications of associating certain circulating, free nucleic acids in the plasma of a patient with the presence of tumors and/or cancer. For example, U.S. Patent Publication No. 2005/0069931 (Mar. 31, 2005) discloses the use of antibodies directed against specific histone N-terminus modifications as diagnostic indicators of disease, employing such histone-specific antibodies to isolate nucleosomes from a blood or serum sample of a patient to facilitate purification and analysis of the accompanying DNA for diagnostic/screening purposes.

Cell-free fetal DNA has been recently shown to exist in plasma and serum of pregnant women as early as the sixth week of gestation, with concentrations rising during pregnancy and peaking prior to parturition. Laboratories have shown the utility of circulating fetal DNA as a unique source of genetic material for non-invasive prenatal evaluation of fetal gender, genetic diseases, and aneuploidy through the use of PCR. For example, quantitative measurements of plasma DNA have been used to correlate risk among cases with various pregnancy-related complications. Although strong evidence exists to support the proposition that elevated levels of maternal plasma DNA may predict development of pre-eclampsia in women prior to onset of disease symptoms, investigators appear to find variable differences between normal control populations, compared to affected cases. Such variability may likely be due to poor plasma DNA recovery and/or inefficiency in PCR as result of impure DNA material. As a consequence, it is felt that transition of this potentially valuable diagnostic tool to a clinical setting for fetal DNA analysis has been predominantly hindered by the fact that abundant amounts of maternal DNA is generally concomitantly recovered along with the fetal DNA of interest. Such of course interferes with obtaining sensitivity in fetal DNA quantification and mutation detection.

Accordingly, the search has gone on for simple, straightforward and more accurate screening/diagnostic tests for fetal chromosomal abnormalities that can be non-invasively performed on a pregnant woman, preferably in the first trimester.

### SUMMARY OF THE INVENTION

Cell-free fetal DNA exists in plasma of pregnant women and is a potentially valuable source of genetic material not only for non-invasive detection of single gene mutations but also for detection of fetal chromosomal aneuploidy. As indicated above, current clinical applications have been limited, given the fact that the overall quality and quantity of fetal DNA isolated is from maternal blood has been highly variable, which variability may very likely be a result of inefficient DNA recovery methods.

It is believed that circulating fetal DNA is predominantly associated with nucleosomes and has a molecular structure distinct from maternal DNA. As a result, such distinctions can be used to achieve isolation and/or enrichment of fetal DNA from maternal plasma, which will then provide an invaluable source of fetal genetic material for non-invasive prenatal diagnosis. It has now been found that a blood sample obtained from a pregnant woman can be tested to screen for the likelihood of Down syndrome (based on DNA quantification) and other related chromosomal abnormalities (e.g., single gene mutations) in a fetus in a straightforward manner with substantially increased accuracy of result.

Heretofore, these approaches have been limited to detection of unique, paternally derived sequences, particularly the Y-chromosome. Maternal mutations inherited by the fetus have been more difficult to detect because both maternal and fetal DNA are often recovered simultaneously, and as a result, the DNA sequences may be indistinguishable. Given that DNA and/or chromosomal abnormalities can be inherited from either the mother or father, the ability to molecularly distinguish fetal DNA from maternal DNA would enable enrichment of fetal DNA in a sample with the result that universal testing of both maternally and paternally derived genetic alterations would then be possible using that isolated DNA.

The amount of circulating DNA has been shown to be directly associated with the measurement of circulatory nucleosomes, and it has been shown that nucleosomes are often packed into apoptotic bodies and phagocytosed by macrophages or neighboring cells. In situations of enhanced cell death, these mechanisms become overloaded, with the result that some nucleosomes are often released into circulation. Nucleosomes are elementary units of chromatin formed by a core of 146 base pairs of DNA wrapped around an octamer of four different histone proteins, and they exist in a variety of forms that contribute to the definition of distinct functional domains within the nucleus. A nucleosome core is connected by linker DNA that varies in length, and such variation is believed to be important for the diversity of gene regulation. Under physiological conditions, such as cells entering into the apoptosis pathway, endonuclease digestion of exposed DNA linker regions between nucleosomes in chromatin occurs; however, the 146 base pairs of DNA around a histone core appear to be conformationally protected from digestion so that stable DNA fragments do exist in circulation. Preliminary data shows that cell-free fetal DNA in plasma is fragmented; however, there is also evidence to support the presence of nucleosomes (DNA bound to histones) and apoptotic bodies in maternal plasma. Moreover, the fact that mononucleosomal units in plasma contain ˜140 bp of DNA further supports the likelihood that circulating fetal DNA is of apoptotic origin.

There are five histone types which are designated HI, H2A, H2B, H3 and H4. Histones can form all manners of protein aggregates both individually and in mixture with one another. There are three major groups of histone genes: (1) replication-dependent histone genes, expression of which is restricted to the S-phase of the cell cycle; (2) replication-independent histone genes or replacement histone genes, which are synthesized independently from DNA replication at constant low level throughout the cell cycle and which are mainly expressed in differentiated or quiescent cells; and (3) tissue-specific histone genes, such as the testis histones. Replacement histone genes differ from S-phase histone genes in their location (i.e., in solitary outside the large histone gene clusters on chromosomes 1 and 6). Also, they have 5′ and 3′ UTR with polyadenylated transcripts, and they are interrupted by introns in their gene structure. It has now been found that histone H3 subtype can be targeted to distinguish fetal DNA from maternal DNA in maternal plasma.

The H3 subtype family consists of four different protein subtypes: the main types (H3.1 and H3.2); the replacement subtype (H3.3); and the testis specific variant (H3t). Although H3.1 and H3.2 are closely related, only differing at Ser⁹⁶, H3.1 differs from H3.3 in at least 5 amino acid positions. H3.1 is highly enriched in fetal liver, in comparison to its presence in adult tissues including liver, kidney and heart. In adult human tissue, the H3.3 variant exceeds the H3.1; whereas the converse is true for fetal liver. It has now been found that the conformational structure of fetal DNA in nucleosomes is such that H3.1 subtype is better exposed, compared to the corresponding H3.1 subtype in maternal nucleosomes and that there are post-translational modifications, e.g., methylation, which occur differently in fetal and maternal histones of H3 subtype, and as a result, this difference can be exploited to target H3.1 histone to enrich and/or isolate fetal DNA based on nucleosome recovery. For example, it is feasible to employ antibodies targeted to a unique exposed section of the fetal H3.1 histone to identify and sequester nucleosomes in maternal plasma which carry fetal DNA; such permits subsequent screening for and/or diagnosis of chromosomal abnormalities via molecular DNA sequencing and/or polymorphic DNA sequence analysis.

In a particular aspect, the invention provides a method for determining a pregnant woman's risk of carrying a fetus with Down syndrome or other fetal chromosome aneuploidy or of a pregnancy complication, which method comprises obtaining a blood plasma sample from the pregnant woman during the first trimester, the second trimester or the third trimester of pregnancy; treating said maternal plasma sample to provide a fraction enriched in fetal DNA as a result of selection based on nucleosome and histone conformational structure; and subjecting said fraction enriched in fetal DNA to analysis.

### DETAILED DESCRIPTION OF THE PREFERRED EMBODIMENTS

DNA is packaged in chromatin. In inactive chromatin, the DNA is complexed to histones and forms nucleosomes. A nucleosome is an octomer of four pairs of histones 2A, 2B, 3 and 4, around which two superhelical turns of 146 bp dsDNA are wound. Histone 1 (H1) and a linker of 60 bp dsDNA connects the individual nucleosomes like beads on a string. During apoptosis, oligo- and mononucleosomes are generated by interchromosomal cleavage of chromatin. Nucleosomes may then be incorporated along with other nuclear components into apoptotic bodies, and in vivo, these bodies are released into the circulation and are cleared by various mechanisms. Efficient clearance prevents the occurrence of nucleosomes in plasma. Thus, their mere presence and abundance in maternal plasma suggests that the situation may be such that the clearance mechanism is either not closely regulated or is simply overwhelmed during pregnancy. Antibody binding to histones can be assayed by commercial ELISA kits; such will allow fluorescent detection and enable analysis of low protein levels (10 to 50 pg/microplate). It appears that fetal DNA is stabilized by the core histones which remain bound to chromatin. Targeting a structural difference between fetal and maternal histones makes enrichment of fetal DNA possible. Fetal DNA, recovered after enrichment, can then be quantified by real-time PCR. Quantification of the various molecular forms of fetal DNA can be determined based on the number of copies of Y-chromosome detected within each possible form (i.e., single stranded and double stranded form of fetal DNA).

Generally, there is provided a diagnostic test for fetal chromosomal abnormalities that involves treating maternal blood to isolate fetal DNA bound to nucleosomes based on chromatin structures; antibodies (Abs) directed to a unique exposed histone section are used to isolate nucleosomes from maternal blood. Such antibodies are created to target a unique, exposed histone peptide sequence that is a characteristic associated with fetal DNA that is present in maternal blood, but which sequence of the corresponding maternal histone is not similarly exposed in the maternal nucleosome. The targeted fetal DNA in the nucleosome complex in maternal blood can then be isolated by immunoprecipitation using one or more of such histone-specific antibodies which attach to such unique exposed sections. Alternatively, such histone-specific antibodies can be linked to a solid support and used to sequester cell-free fetal DNA in nucleosome complexes from a maternal blood sample. Such a support may be in particulate form, or it could be a plate, beads, a filter, a membrane or a microflow device. Methods for attaching antibodies to insoluble supports are well known to those skilled in this art. After a blood sample has been in contact with such histone-specific antibodies under conditions suitable to promote specific binding of the antibody to its target antigen, the sequestered fetal DNA in the nucleosome complexes having such a targeted exposed histone sequence can be isolated using standard techniques known to those skilled in the art.

Once the targeted fetal DNA in the nucleosome complexes have been isolated from the maternal blood sample, the DNA associated with the nucleosomes can be recovered using standard techniques known in this art. For example, the DNA can be released, and the recovered DNA can then optionally be amplified through PCR, Real Time PCR, Quantitative Fluorescent PCR (QF-PCR) or by another suitable amplification technique, such as whole genome amplification. Once DNA associated with the immunoprecipitated or otherwise recovered nucleosomes has been purified, the genes encoded by that DNA can be identified and analyzed, as by mutation microarrays or chromosome gene-specific microarrays.

Generally, the steps used to identify the genes encoded by the fetal DNA associated with the isolated fetal nucleosomes can include any of the analytical procedures known to those skilled in this art; for example, gene sequences can be identified by direct microsequencing of the purified fetal DNA. Alternatively, the purified fetal DNA can be first amplified using Real Time PCR and then subjected to sequence analysis or to oligonucleotide microarrays.

Genes encoded by the amplified fetal DNA associated with the isolated nucleosomes can also be identified by contacting the purified fetal DNA with known nucleic acid probes under conditions suitable for hybridization of complementary sequences; hybridization of the purified DNA to its complement probe constitutes identification of that gene. Such nucleic acid probes can be labeled with a detectable marker using standard techniques known to those skilled in this art; for example, nucleic acid probes can be labeled with a fluorophore, a radioisotope, or a non-isotopic labeling reagent such as biotin to facilitate detection.

Known nucleic acid sequences representing various gene abnormalities of interest are often immobilized on a solid surface, preferably in the form of a microarray. Thus, a signal generated at a specific region on such surface as a result of hybridization of a purified fetal nucleosome DNA sequence to its complement serves to identify the presence in the sample of the gene encoded by that sequence.

As earlier indicated, it has been found that histone H3 subtype can be targeted to distinguish fetal DNA from maternal DNA, both of which will be present in circulating maternal plasma. It appears that, in the fetal liver, the H3.1 variant is highly enriched and significantly exceeds the H3.3 variant; this is the opposite of human liver tissue so its presence in greater relative quantity can be used to detect fetal DNA. Moreover, it has been found that the conformational structure of fetal DNA is such that portions of the H3.1 subtype are better exposed in the fetal nucleosome, compared to the same subtype in maternal nucleosomes and that there are post-translational modifications that are different. Such a morphological difference, for example, allows this exposed section of H3.1 to be used to select for fetal nucleosomes and their associated DNA. Preferably, a peptide sequence is chosen that will not be similarly exposed in the comparable maternal nucleosome, and such a sequence can be chosen based upon the conformational structure of fetal DNA and histones. More preferably, a unique peptide sequence is selected that is not present in other histones and will be exposed. The difference of five amino acids between the H3.1 and H3.3 proteins is best exploited to facilitate the isolation of fetal DNA from maternal blood. More specifically, there is a unique 10 amino acid sequence present near the C-terminus in the H3.1 subtype which distinguishes it from the H3.3 subtype, which sequence is part of the histone that is exposed in the fetal nucleosome. Thus, hybridization to this sequence by antibodies that are so targeted can be used to isolate fetal nucleosomes and the associated fetal DNA.

Histone-specific antibodies (Abs) raised against the decapeptide; Ser-Ala-Val-Met-Ala-Leu-Gln-Glu-Ala-Cys are preferred, which will hybridize to the targeted unique exposed peptide section of H3.1. However, larger peptide or smaller peptides which include a significant unique part of this sequence might alternatively be used. If such antibodies, for example, contain biotin, they may be sequestered using avidin conjugated to any desirable label, such as a fluorochrome. Alternatively, these histone-specific antibodies can be sequestered through the use of a secondary antibody, which is labeled and is specific for the primary antibody. As a further alternative, the histone-specific antibody may be directly labeled with a radioisotope or a fluorochrome, such as FITC or rhodamine, so such secondary detection reagents would not be required. Selective separation of these fetal DNA-origin histone complexes is then effected by immunoprecipitation using one of the methods mentioned above.

Once separated, washing using suitable buffer solutions as well known in this art is then used to eliminate non-specifically bound biological material that would be present in the maternal blood to provide a highly enriched fraction comprising histones of fetal origin and the associated fetal DNA. Analysis can then be carried out using molecular diagnostics. For example, the fetal DNA can be released from complexes with the antibodies used in the isolation, collected, and then subjected to Real Time PCR where pairs of primers are provided. Ultimate treatment of the resultant products from such Real Time PCR amplification of the fetal DNA is then carried out in a manner known in this art. For example, one such method of analysis to detect for chromosomal disorders is described in pending U.S. Patent Application Publication No. 2005/025011 published Nov. 10, 2005 (Detection of Chromosomal Disorders), the disclosure of which is incorporated herein by reference. The primers used may have labels incorporated therein as is known in this art and described in that application.

As a part of such analysis, it may be desirable to first confirm that the enriched/isolated DNA is substantially all of fetal origin, prior to running diagnostic tests for genetic disorders or the like. Such testing might be carried out for the presence of the Y-chromosome, and if present, assurance that the DNA is substantially all fetal could be obtained through quantitative testing for this and another ubiquitous sequence. Polymorphic DNA sequence analysis could then be used for determination of parental origin to confirm fetal DNA was isolated.

Other such methods of detection can utilize microarrays such as those described in U.S. Pat. No. 6,174,683 or published U.S. Patent Application No. 2004/0029241, the disclosures of which are incorporated herein by reference.

The following example is presented to provide the best mode presently known for carrying out the invention using antibodies to sequester the fetal nucleosomes.

A 10-20 ml liquid blood sample is obtained from a pregnant woman early in the second trimester (or late in the first trimester) of pregnancy. The blood sample is collected in vacutainers containing an anti-coagulant (i.e., ACD, EDTA or sodium heparin). The whole blood is processed to separate plasma from the cellular layer by centrifugation. The recovered plasma is subjected to filtration (using 0.22μ filter) and may then be frozen at −80° C. for future DNA extraction if desired.

When extraction is ready to be begun, a microflow device of the type disclosed in U.S. patent application Ser. No. 11/038,920, filed Jan. 18, 2005, having a collection region with a multitude of randomly positioned posts is prepared by attaching histone-specific antibodies within its post-containing collection region. The antibodies are designed to couple with a unique decapeptide which is present in a region near the C-terminus of histone 3.1. The region selected, which is exposed to a far greater extent in fetal nucleosomes than in maternal nucleosomes, contains the following amino acid residue sequence: Ser-Ala-Val-Met-Ala-Leu-Gln-Glu-Ala-Cys.

The blood sample, prepared as above, is supplied to the thus-prepared microflow device and caused to slowly travel therethrough, drawn by a vacuum pump. The Abs couple to and sequester chromatin in the maternal plasma containing fetal histone 3.1. by coupling to the exposed unique sequence. The microflow device is then washed with buffers, and washing is repeated 2-3 times to remove nonspecifically bound biologic material from the maternal plasma.

The biological material sequestered by the antibodies is released from the microflow device and subjected to purification to remove salts that might be present. It is then concentrated to an appropriate volume, i.e., 20-50 μl and analysis is then carried out to detect Y-specific sequences using Realtime PCR or fluorescence-based PCR. The results of the analysis show positive detection of male Y-sequences in the DNA, which is evidence of fetal DNA being present in the biological material that is sequestered by the antibodies. The non-presence, to any significant extent, of maternal DNA in the DNA material that is sequestered is next shown using quantitative Realtime PCR of two loci. For example, showing that Y-levels are equal to those of a second ubiquitous sequence (e.g., β-globin), is evidence that most or nearly all of DNA is of fetal origin. Alternatively, a determination of the relative proportion of H3.1 to H3.3 in the DNA may be used to show that the relative proportions which exist are such as would be present in nucleosomes of fetal origin, which would confirm the fetal origin of the DNA.

Following such confirmation, analysis of the DNA for aneuploidies or other genetic disorders or conditions that might suggest pregnancy complications may be carried out with confidence. The isolated fetal DNA fraction may be subjected to molecular DNA sequencing or polymorphic DNA sequence analysis. Such analysis may utilize Realtime PCR to quantify DNA levels that are associated with specific DNA sequences to screen for aneuploidies or may use it or PCR to amplify the DNA and then incubate it with mutation microarrays or gene-specific microarrays.

Although the invention has been described in terms of the best mode presently known for carrying out the invention, it should be understood that various changes and modifications as would be obvious to one skilled in this art may be made without departing from the scope of the invention which is set forth in the claims appended hereto.

1. A method for isolating fetal DNA comprising isolating DNA from a chromatin component of a maternal host sample, wherein the chromatin component is isolated based on at least one of its features that is present substantially in a fetus, but not in a maternal host.

2. The method of claim 1, wherein the chromatin component consists substantially of nucleosomes.

3. The method of claim 1, wherein the maternal host sample is a blood, plasma, serum, saliva, or urine sample.

4. The method of claim 1, wherein the chromatin component is isolated based on at least one of its structure features associated with histone H3 subtype.

5. The method of claim 1, wherein the chromatin component is isolated based on at least one of its structure features associated with histone H3.1 subtype.

6. The method of claim 1, wherein the chromatin component is isolated based on a section of H3.1 that is more exposed in fetal nucleosomes than in maternal nucleosomes.

7. The method of claim 1, wherein the chromatin component is isolated based on at least one of its features associated with histone H3.1, but not with histone H3.3.

8. The method of claim 1, wherein the chromatin component is isolated by using an antibody that specifically binds to an epitope associated with a fetal chromatin structure, but not a maternal chromatin structure.

9. The method of claim 1, wherein the chromatin component is isolated by using an antibody that specifically binds to an epitope comprising an amino acid sequence of Ser-Ala-Val-Met-Ala-Leu-Gln-Glu-Ala-Cys.

10. A method of conducting a genetic test for a pregnant woman comprising isolating DNA from a chromatin component of a sample obtained from the pregnant woman, wherein the chromatin component is isolated based on at least one of its features that is present substantially in a fetus, but not in a maternal host.

11. The method of claim 10, wherein the sample is obtained during the first, second, or third trimester or a combination thereof.

12. The method of claim 10, further comprising using the isolated DNA to conduct a molecular genetic test.

13. The method of claim 10, further comprising using the isolated DNA to conduct a quantitative molecular genetic test based on a PCR assay or Realtime PCR assay.

14. The method of claim 10, wherein the genetic test is a diagnostic test for a genetic disorder.

15. The method of claim 10, wherein the genetic test is a diagnostic test for a maternally or paternally derived genetic disorder.

16. An antibody specifically binds to an epitope comprising an amino acid sequence of Ser-Ala-Val-Met-Ala-Leu-Gln-Glu-Ala-Cys.

17. A kit comprising an antibody that specifically binds to an epitope comprising an amino acid sequence of Ser-Ala-Val-Met-Ala-Leu-Gln-Glu-Ala-Cys.

18. A kit comprising an antibody that specifically binds to an epitope comprising an amino acid sequence of Ser-Ala-Val-Met-Ala-Leu-Gln-Glu-Ala-Cys, wherein the antibody is provided on a solid support.

19. A kit comprising an antibody that specifically binds to an epitope comprising an amino acid sequence of Ser-Ala-Val-Met-Ala-Leu-Gln-Glu-Ala-Cys, wherein the antibody is provided on a solid support of a microchannel.

20. The kit of claim 17 further comprising an instruction for using the antibody to isolated fetal DNA from a maternal sample.
